(19) United States
(12) Patent Application Publication
(10) Pub. No.: US 2020/0253743 A1
(43) Pub. Date: Aug. 13, 2020

(54) SPINAL IMPLANT DEVICE FOR HERNIATED DISCS

(71) Applicant: GLOBUS MEDICAL, INC., Audubon, PA (US)

(72) Inventor: Kurt Faulhaber, Renton, WA (US)

(21) Appl. No.: 16/274,459

(22) Filed: Feb. 13, 2019

(51) Int. Cl. A61F 2/44; A61B 17/70; A61B 17/80

(52) U.S. Cl. CPC A61F 2/442; A61B 2017/00261; A61B 17/8028; A61B 17/70

(57) ABSTRACT

Embodiments are directed to a spinal implant device for reducing effects of a spinal disc herniation that includes a support frame, where the support frame has a first end, a second end, and a mounting hole for receiving a fastener that attaches the support frame to a vertebra, and a cushion attached to the support frame. Embodiments are also directed to a system for implanting a medical device to a spine that includes a spinal implant device having a support frame, where the support frame has a first end, a second end, and a mounting hole, and a cushion attached to the support frame. The system also includes a fastener for attaching the spinal implant device to a vertebra, where the spinal implant device is moveable to align the cushion to a disc space created after a portion of a herniated disc is removed during surgery.

### BACKGROUND

A common medical problem is a herniated disc located between two vertebrae of a spine. The herniated disc is often the result of the spinal disc being pushed out or squeezed between vertebrae, causing the disc to expand and stretch through a tear in the outer covering of the disc. A herniated disc, also known as a slipped disc or ruptured disc, affects nearby nerves and results in pain, weakness, and numbness in the back and extremities. Herniated discs are often the result of a gradual degradation of the disc material over time. Other contributing factors include spinal damage due to arthritis, the occupation of the patient, and accidents.

In some cases, a herniated disc does not require surgery. However, surgery is often required to alleviate the disc rupture and relieve the pain. During surgery, portions of the herniated disc may be cut away and creating an empty space where the disc once occupied. For patients with multiple herniated disc incidents, a spinal fusion procedure is sometimes recommended to prevent further incidents. Surgery may involve removing the entire disc and fusing the two adjacent vertebrae together. However, patients with fused spines often experience limited motion and, in some cases, a stooped appearance. What is needed is a device to replace the disc material between vertebrae to restore full functionality of the disc while minimizing the risk of future disc ruptures, thus reducing the need for additional surgeries and benefiting patients with less pain and fewer surgeries.

### SUMMARY

In an exemplary embodiment, the present disclosure provides a spinal implant device for reducing effects of a spinal disc herniation that includes a support frame, where the support frame has a first end, a second end, and a mounting hole for receiving a fastener that fixably attaches the support frame to a vertebra, and a cushion attached to the support frame.

In another exemplary embodiment, the present disclosure provides a system for implanting a medical device to a spine that includes a spinal implant device having a support frame, where the support frame has a first end, a second end, and a mounting hole, and a cushion attached to the support frame. The system also includes a fastener receivable through the mounting hole for fixably attaching the spinal implant device to a vertebra, where the spinal implant device is positionable to align the cushion to a disc space created after a portion of a herniated disc is removed during surgery.

In an exemplary embodiment, the present disclosure provides a method for reducing the effects of a spinal disc herniation that includes removing a portion of a spinal disc to create a disc space, positioning a spinal implant device against an outer surface of a vertebra adjacent to the spinal disc such that a cushion of the spinal implant device is aligned with the disc space, where the cushion is attached to a support frame of the spinal implant device, and affixing the spinal implant device to the vertebra.

### BRIEF DESCRIPTION OF THE DRAWINGS

These drawings illustrate certain aspects of some examples of the present invention, and should not be used to limit or define the invention, wherein:

FIG. 1 illustrates an example section of a spinal column with a herniated spinal disc interposed between two vertebrae.

FIG. 2 illustrates the example section of the spinal column of FIG. 1 with a herniated spinal disc after surgery to remove portions of the disc.

FIGS. 3 and 4 illustrate a front isometric view of an example spinal implant device having a cushion and two arms.

FIG. 5 illustrates an example spinal implant device having an attachment hole and fastener.

FIG. 6 illustrates an example spinal implant device having a main body with attachment holes and a cushion attached to an arm.

FIG. 7 illustrates an example spinal implant device having a monolithic body with attachment holes and multiple cushions.

FIGS. 8, 9, 10, and 11 illustrate an example spinal implant device positioned onto a vertebra and a disc space.

### DETAILED DESCRIPTION

Embodiments are directed to a spinal implant device for reducing effects of a spinal disc herniation that includes a support frame, where the support frame has a first end, a second end, and a mounting hole for receiving a fastener that attaches the support frame to a vertebra, and a cushion attached to the support frame.

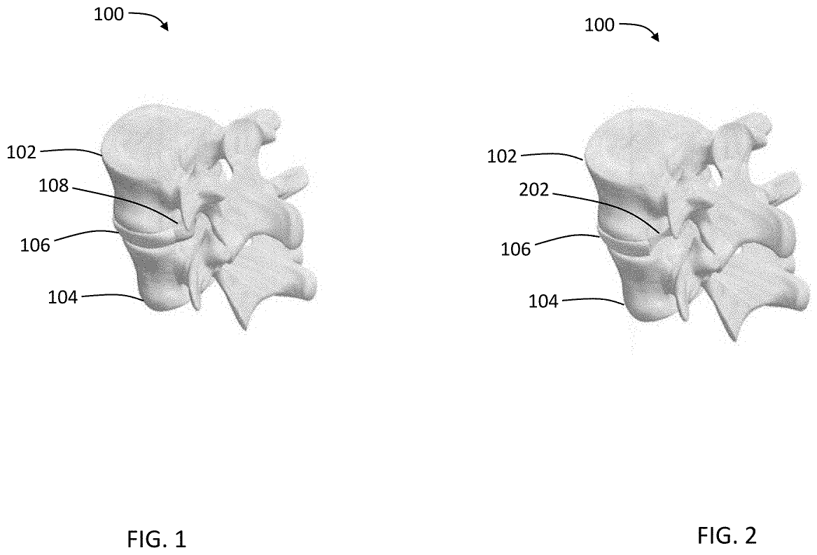

FIG. 1 illustrates an example section of a spinal column 100 with a herniated spinal disc 106 interposed between a first vertebra 102 and a second vertebra 104. The first vertebra 102 and the second vertebra 104 are referred to herein collectively as vertebrae 102, 104 and individually as first vertebra 102 and second vertebra 104. As illustrated, the herniated spinal disc 106 may include a hernia region 108 where a mass of disc material extends beyond the vertebrae 102, 104 in a radial direction away from a center of the spinal column 100. The herniated spinal disc 106 comprises a soft inner material and a firm but flexible outer surface. Spinal discs work as connective tissue between adjacent vertebrae and absorbs shock to the spine while maintaining flexibility between joints. In some instances, a patient undergoes trauma to the spinal area that results in the herniated spinal disc 106. In such instances, the hernia region 108 can be treated so that pain and additional potential damage to the spinal column 100 is minimized.

FIG. 2 illustrates the example section of the spinal column 100 of FIG. 1 with the herniated spinal disc 106 after performing a surgical procedure to remove portions of the herniated spinal disc 106 to form a disc space 202, in accordance with example embodiments. In some embodiments, a medical professional can remove portions of the herniated spinal disc 106 that extends beyond typical boundaries within the spinal column 100 to form an empty region or disc space 202. The disc space 202 is an open area between two vertebrae 102, 104 where the herniated spinal disc 106 once occupied. Left unfilled, the disc space 202 causes the vertebrae 102, 104 to have less cushioning which can lead to a greater likelihood of reoccurrence of a herniated disc condition and further discomfort and pain for the patient. Furthermore, additional surgery may be required, encompassing additional burdens and risks for the patient.

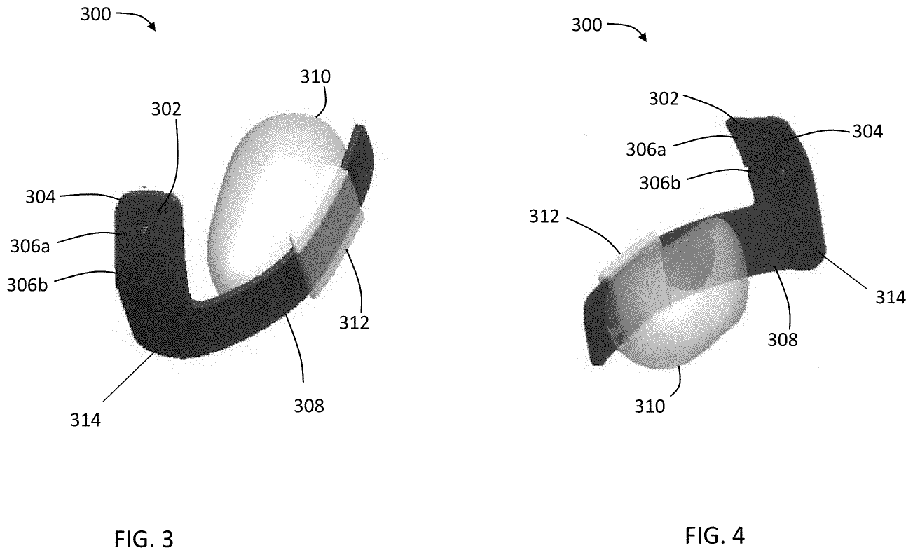

FIGS. 3 and 4 illustrate a first spinal implant device 300 comprising a support frame 302 with a first arm 304, a second arm 308, and a cushion 310. The first arm 304 and the second arm 308 may be configured to retain mechanical energy and operate as a spring by attempting to return to their original shape after mechanical deflection such as experienced during movement of the patient and the spinal column 100. In some embodiments, the first spinal implant device 300 is known as a disc herniation dam device. Without limitation, the support frame 302 is comprised of suitable materials including at least one of metals, nonmetals, polymers, composites, ceramics, and/or combinations thereof. Support frame 302 can be of any suitable size, height, and/or shape. Without limitation, a suitable shape includes, but is not limited to, cross-sectional shapes that are flat, circular, elliptical, triangular, rectangular, square, hexagonal, and/or combinations thereof. In some embodiments, the support frame 302 comprise a band. In some embodiments, support frame 302 comprises a hollow, elongated tubular. In some embodiments, support frame 302 has a length in a range of from about 1 centimeter to about 10 centimeters. In some embodiments, support frame 302 has a width of about 1 millimeter to about 50 millimeters. However, the scope of the disclosure is not so limited to these dimensions for the support frame 302. Rather, support frame 302 can have any suitable dimensions as desired for a particular application.

The first spinal implant device 300 further comprises one or more attachment holes 306a, 306b and a cushion attachment mechanism 312. In operation, the first spinal implant device 300 may be positioned so that the attachment holes 306a, 306b are proximate to a vertebra such as first vertebra 102 or second vertebra 104 of FIG. 1, in accordance with present embodiments. Cushion attachment mechanism 312 may use any suitable technique for coupling the cushion 310 to the support frame 302. By way of example, the cushion attachment mechanism 312 may comprise at least one of clips, screws, washers, nuts, bolts, pins, sockets, studs, adhesives, or other suitable devices. Other embodiments are possible and are not limited by this example. In some embodiments, the attachment holes 306a, 306b may be formed by drilling or punching the support frame 302. The attachment holes 306a, 306b can also be fabricated by a suitable process to produce through-holes to allow a mechanical fastener to affix the first spinal implant device 300 to the first vertebra 102 and/or the second vertebra 104. In addition, the first spinal implant device 300 may be positioned to place the cushion 310 proximate to, and substantially filling, the disc space 202 of FIG. 2, in accordance with example embodiments. Thus positioned, the first spinal implant device 300, and the cushion 310, may replace portions of the herniated spinal disc 106 and add cushioning, stability, and support to the adjoining vertebrae 102, 104 to reduce the negative effects of a disc rupture condition.

In some embodiments, the cushion 310 comprises a flexible material to emulate spinal disc material. In some embodiments, the cushion 310 comprises a flexible container. The container may be considered flexible as it may be manufactured from a material capable of confirming to its surrounding at pressures encountered in the disc space 202 (e.g., shown on FIG. 2). For example, the flexible container may comprise a synthetic material. In some embodiments, the cushion 310 may comprise a saline solution suspended in a flexible container. In some embodiments, the cushion 310 may comprise a silicone gel suspended in a flexible container. In some embodiments, the material in the flexible container may be an incompressible fluid. The cushion 310 may have any suitable volume for occupying the disc space 202 as described herein. In some embodiments, the cushion 310 may have a volume of about 0.5 cm³ to about 30 cm³. As previously described, embodiments may use the cushion attachment mechanism 312 for coupling the cushion 310 to the first spinal implant device 300.

In some embodiments, the support frame 302 further comprises a bend 314 that is attached to the first arm 304 and the second arm 308. As illustrated, the first arm 304 and the second arm 308 may be jointed at the bend 314. The bend 314 may be at any suitable angle and allows the first arm 304 and the second arm 308 to be oriented to each other to both allow the first spinal implant device 300 to be attached to the first vertebra 102 or the second vertebra 104 and to position the cushion 310 over the disc space 202. For example, the bend 314 may be configured to position the first arm 304 at an angle with respect to the second arm 308 of about 40° to about 150°, about 60° to about 120°, or about 85° to about 95°. In some instances, the angle may be about 90°. With the bend 314, the first spinal implant device 300 thus allows the first arm 304 to flex along as first axis and the second arm 308 to bend along a second axis that is different than the first axis. In some embodiments, the first arm 304 and the second arm 308 are fabricated having different spring rates, thus allowing, for example, the first arm 304 a spring rate corresponding to reacting to the motion of the spinal column 100 movement while the second arm 308 has a spring rate corresponding to reacting to motion of the herniated spinal disc 106. Other embodiments are possible and are not limited by this example.

In some embodiments, the support frame 302 is comprised of a memory metal (also known as a shape-memory alloy) that remembers its original shape after being deformed by an external mechanical force such as from spinal column 100 (e.g., shown on FIG. 1) movement. After being subject to the mechanical force, the memory metal attempts to return to its pre-deformed shape. In some embodiments, the support frame 302 is comprised of a polymer material that retains its shape after deformation. In some embodiments, the support frame 302 is made of any suitable materials to include metals, nonmetals, polymers, composites, ceramics, and/or combinations thereof. Other materials are possible and are not limited by these examples. Support frame 302 is fabricated to any suitable size, height, and/or shape to conform to different sizes of vertebrae 102, 104. In some embodiments, the support frame 302 may include the bend 314 that is a left-hand bend, while in other embodiments the support frame 302 may include the bend 314 that a right-hand bend.

In some embodiments, more than one of the first spinal implant device 300 may be fixably attached to one or more of the vertebrae 102, 104. The need for multiple first spinal implant devices 300 are caused by multiple herniated regions 108 of the herniated spinal disc 106, multiple herniated spinal discs 106, or a herniated spinal disc 106 that is physically large and requires multiple cushions 310 to correct.

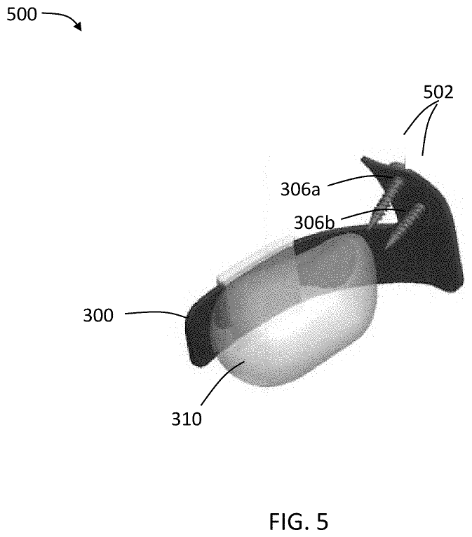

FIG. 5 illustrates a first spinal implant device 300 having multiple attachment holes 306a , 306b and two fasteners 502 to form a system 500 for implanting a medical device to a spine. Fastener 502 may utilize any suitable technique for securing the first spinal implant device 300 to at least one of the vertebrae 102, 104 (e.g., shown on FIG. 1). By way of example, fastener 502 may comprise at least one of clips, screws, washers, nuts, bolts, pins, sockets, studs, or adhesives, or other suitable fastening device. In some embodiments, the first spinal implant device 300 is installed using clips or adhesives. Other embodiments are possible and are not limited by this example. In some embodiments, multiple fasteners 502 may be used in conjunction with multiple attachment holes 306a, 306b . In some embodiments, different types of fasteners 502 are used, while in other embodiments, fasteners 502 of a single style are used. In one embodiment, the fastener 502 is a surgical screw that screws into one or more drilled attachment holes 306a, 306b in the at least one of the vertebrae 102, 104. During a surgical procedure, embodiments may include positioning the first spinal implant device 300 against at least one of the vertebrae 102, 104. In some embodiments, the position for the receiving holes in the at least one of the vertebrae 102, 104 may be marked. Embodiments may then include a medical professional drilling the receiving holes into the vertebra 102. Next, embodiments may include the medical professional setting the first spinal implant device 300 into place and installs one or more fasteners 502 (e.g., surgical screws) through the attachment holes 306a, 306b and into the receiving holes, which may be done using hand and/or powered tools. In some embodiments, the fasteners 502 may be permanently installed in at least one of the vertebrae 102, 104 to affix the first spinal implant device 300 in alignment with the spinal disc 106 and the disc space 202. FIG. 5 illustrates the use of two fasteners 502 to install the first spinal implant device 300, but in some embodiments, more or fewer fasteners 502 are used. In some embodiments, the first spinal implant device 300 uses a single attachment hole 306a and fastener 502 for fastening the first spinal implant device 300 to the vertebra 102.

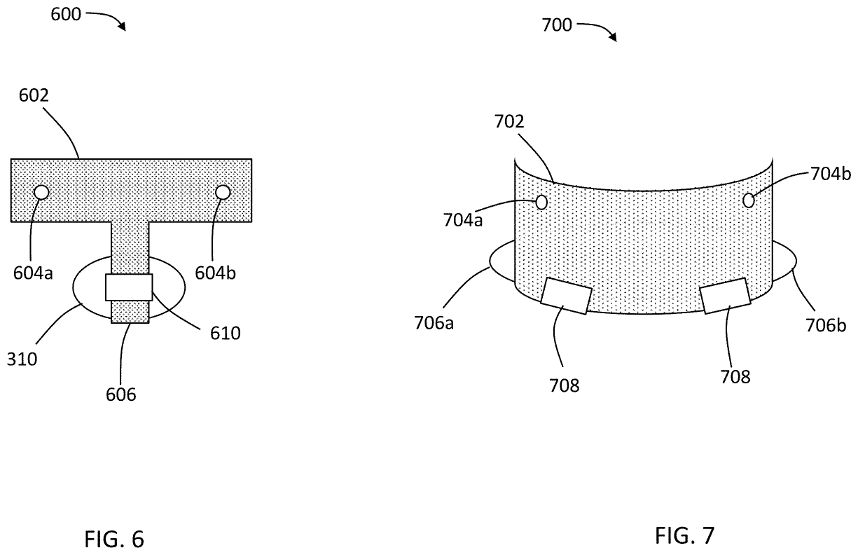

FIG. 6 illustrates a second spinal implant device 600 comprising a main body 602 with attachment holes 604a, 604b, an arm 606, and a cushion 310 attached to the arm 606 with a cushion attachment mechanism 610. In some embodiments, the main body 602 may comprise a plate. As illustrated, the arm 606 may extend from a central portion of the main body 602. However, it is not necessary for the arm 606 to extend from the central portion and embodiments may extend from other portions of the main body 602. The cushion 310 of second spinal implant device 600 may be similar to the cushion 310 of FIG. 3 in form and function. The cushion attachment mechanism 610 may utilize any suitable technique for attachment of the cushion 310 to the arm 606. By way of example, the cushion attachment mechanism 610 may comprise clips, screws, washers, nuts, bolts, pins, sockets, studs, adhesives, or other suitable devices. Other embodiments are possible and are not limited by this example. In some embodiments, the main body 602 may curved corresponding to the curve of a spine, such as vertebrae 102, 104 of FIG. 1. In some embodiments, the main body 602 comprises a memory metal that returns to an original shape after being subject to a mechanical force. In some embodiments, the main body 602 has a spring rate, while in other embodiments, the main body 602 is fixed and the arm 606 has a spring rate that allows the cushion 310 to exert mechanical pressure against the exterior of the disc space 202 of FIG. 2.

Continuing with FIG. 6, the cushion 310 comprises a flexible pad or pillow to allow the cushion 310 to be in direct contact with a spinal disc 108 without causing discomfort or damage. The cushion 310 is positioned so that the disc space 202 is occupied by the cushion 310. The cushion 310 is fixably attached to the arm 606 by any suitable cushion attachment mechanisms 610, including but not limited to clips, screws, washers, nuts, bolts, pins, sockets, studs, or adhesives. In operation, the second spinal implant device 600 is positioned across the vertebra 102 of FIG. 1 and is permanently affixed to the vertebra 102 using fasteners (not shown) that pass through main body attachment holes 604a, 604b. Once in place, the arm 606 holds the cushion 310 in place in and over the disc space 202. As the arm 606 has a spring rate and/or is made of memory metal, the arm allows the cushion 310 to resist mechanical deflection of the vertebrae 102, 104 and/or the spinal disc 106.

The second spinal implant device 600 may be used in any suitable application, including, but not limited to, for situations where the use of the first spinal implant device 300 is not practical or possible. In some embodiments, the second spinal implant device 600 may be used when it is desirable to have only the arm 606 with a spring rate. Other applications are possible and are not limited to this example.

FIG. 7 illustrates a third spinal implant device 700 having a curved plane body 702 with curved plane attachment holes 704a, 704b and multiple cushions 706a, 706b. In some embodiments, cushions 706a, 706b are similar to cushion 310 of FIG. 3 in form and function. While multiple of the cushions 706a, 706b are shown, in some embodiments, the third spinal implant device 700 may only comprise a single large cushion (not shown) that extends the width of the curved plane body 702. In some embodiments, the cushions 706a, 706b are held in position but the use of one or more cushion attachment mechanisms 708. The cushion attachment mechanism 708 may use any suitable technique for attachment of the cushions 706a, 706b. For example, the cushion attachment mechanisms 708 may comprise clips, screws, washers, nuts, bolts, pins, sockets, studs, adhesives, or other suitable devices. Other embodiments are possible and are not limited by this example. In some embodiments, multiple cushion attachment mechanisms 708 are used to attach the cushion to the curved plane body 702.

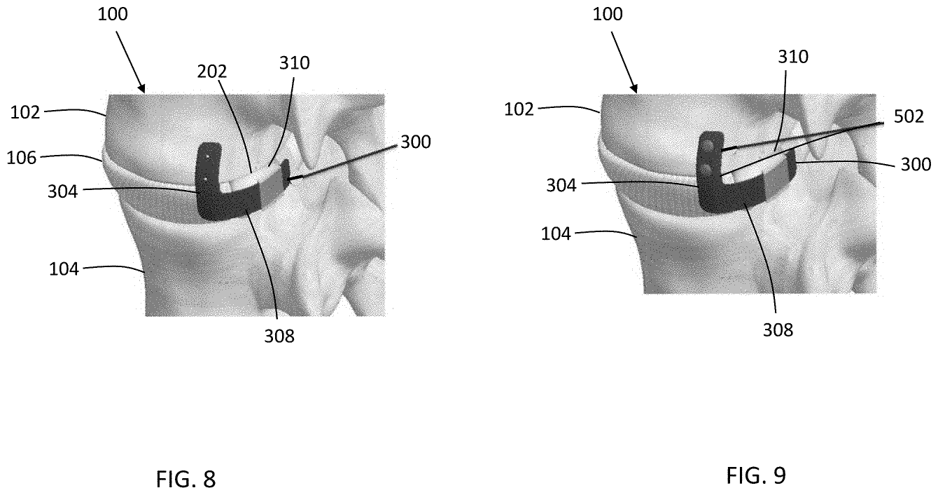

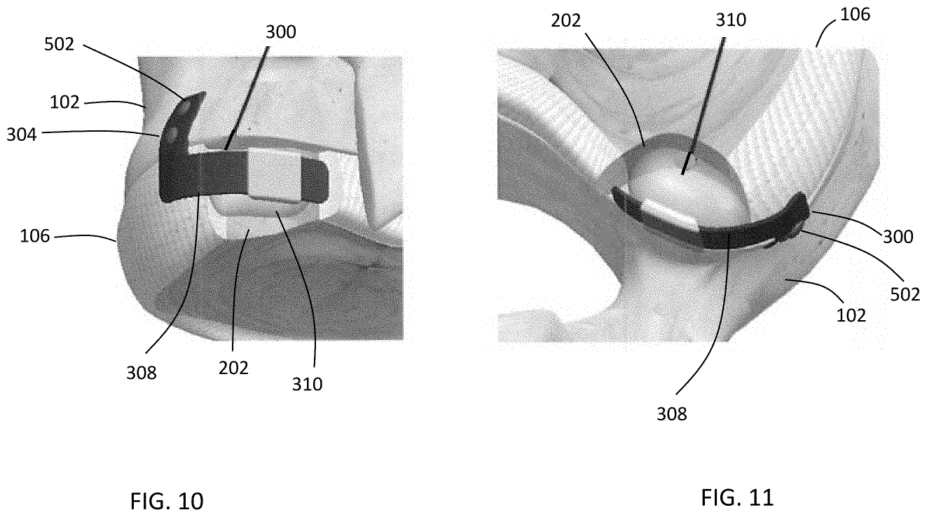

Turning now to FIGS. 8-11, attachment a spinal implant device, such as the first spinal implant device 300, to the first vertebra 102 will now be described in accordance with example embodiments. FIG. 8 illustrates the first spinal implant device 300 in position with second arm 304 adjacent to the first vertebra 102 in accordance with example embodiments. FIG. 9 illustrates the first spinal implant device 300 affixed to the first vertebra 102 using fasteners 502, in accordance with example embodiments. FIGS. 10 and 11 illustrate alternative views of the spinal implant device 300 affixed to the first vertebra 102 using fasteners, in accordance with example embodiments. Turning now to FIG. 8, the herniated spinal disc 106 may located between the first vertebra 102 and the second vertebra 104. As previously described, embodiments may include removing a portion of the herniated spinal disc 106 to form a disc space 202. In operation, embodiments may include positioning the first spinal implant device 300 such that the first arm 304 extends over at least one of the vertebrae 102, 104 and the second arm 308 extends along the herniated spinal disc 106. As illustrated, the first arm 304 may be adjacent to the first vertebra 102 such that the first arm 304 extends over the first vertebra. In the illustrated embodiment, the second arm 304 holds the cushion 202 such that the cushion may be at least partially disposed in the disc space. Referring now to FIG. 9, embodiments may then include fixable attaching the first implant device 300 to the spinal column 100. For example, fasteners 502 may be used to attach the first implant device 300 to the first vertebra 102 of the spinal column 100 with the cushion 310 proximate to the disc space 202. Thus positioned, the cushion 310 may act as an artificial disc between two vertebrae 102, 104 while also holding the disc 106 in place to reduce the risk for further herniation. In some embodiments, the first arm 304 bends under mechanical stress as the spinal column 100 moves and twists. After the spinal column 100 movement ceases, the first arm 304 returns to its original shape. In a similar manner, the second arm 308 bends under mechanical force, and after the mechanical forces are removed, returns to its original shape. Both the first arm 304 and the second arm 308 work to maintain the cushion's 310 position within the disc space 202 so that the cushion 310 can continue to function as an artificial spinal disc.

Embodiments are directed towards a spinal implant device for reducing effects of a spinal disc herniation that includes a support frame, where the support frame has a first end, a second end, and a mounting hole for receiving a fastener that fixably attaches the support frame to a vertebra, and a cushion attached to the support frame.

According to some embodiments, the device may also include where the support frame is made of a memory metal that returns to a known shape after a mechanical deflection. The device may also include where the support frame comprises a polymer material that returns to a known shape after a mechanical deflection. The device may also include where the cushion comprises a container and a saline solution disposed in the container. The device may also include where the cushion comprises a container and a silicone gel disposed in the container. The device may also include where the cushion is formed around the second end of the support frame. The device may also include where the first end curves around a first axis and the second end curves around a second axis. The device may also include where the support frame and the cushion curves around a first axis. The device may also include where the cushion has a volume of about 0.5 cm³ to about 30 cm³. The device may also include where the support frame comprises a first arm having the first end and a second arm having the second end, wherein the first arm and the arm are connected at a bend of the support frame. The device may also include where the support frame is a metal band comprising the first arm and the second arm, wherein the bend positions the first arm and an angle with respect to the second arm of about 60° to about 120°. The device may also include where the spinal implant device comprises a plate having the first end and an arm having the second end and extending from a central portion of the plate, wherein the arm holds the cushion. The device may also include where the spinal implant device comprises a curved plane body having the first end and the second end, wherein more than one of the mounting hole is formed in the curved plane body.

Embodiments are directed towards a system for implanting a medical device to a spine that includes a spinal implant device having a support frame, where the support frame has a first end, a second end, and a mounting hole, and a cushion attached to the support frame. The system also includes a fastener receivable through the mounting hole for fixably attaching the spinal implant device to a vertebra, where the spinal implant device is positionable to align the cushion to a disc space created after a portion of a herniated disc is removed during surgery.

According to some embodiments, the system may also include where the cushion is positioned on the support frame to block expansion of the spinal disc when attached to the spine. The system may also include where the spinal implant device has a spring rate to maintain the position of the cushion in relation to the disc space during movement of the vertebra. The system may also include where the fastener is made of at least one of clips, screws, washers, nuts, bolts, pins, sockets, rods, studs, or adhesives. The system may also include where the cushion comprises a container and an incompressible fluid disposed in the container. The system may also include where the support frame comprises a first arm having the first end and a second arm having the second end, wherein the first arm and the arm are connected at a bend of the support frame.

Embodiments are directed towards a method for reducing the effects of a spinal disc herniation that includes removing a portion of a spinal disc to create a disc space, positioning a spinal implant device against a vertebra adjacent to the spinal disc such that a cushion of the spinal implant device is aligned with the disc space, where the cushion is attached to a support frame of the spinal implant device, and affixing the spinal implant device to the vertebra.

According to some embodiments, the method may also include where affixing the spinal implant device includes forming an attachment hole in the vertebra, positioning the spinal implant device against an outer surface of the vertebra where the mounting hole is axially aligned with the attachment hole, and affixing the spinal implant device to the vertebra with a fastener. The method may also include where the spinal implant device has a spring rate to maintain the position of the cushion in relation to the disc space during movement of the vertebra.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A spinal implant device for reducing effects of a spinal disc herniation, comprising:
a support frame, wherein the support frame comprises a first end, a second end, and
a mounting hole for receiving a fastener that fixably attaches the support frame to a vertebra; and
a cushion attached to the support frame.

2. The device of claim 1, wherein the support frame comprises a memory metal that returns to a known shape after a mechanical deflection.

3. The device of claim 1, wherein the support frame comprises a polymer material that returns to a known shape after a mechanical deflection.

4. The device of claim 1, wherein the cushion comprises a container and a saline solution disposed in the container.

5. The device of claim 1, wherein the cushion comprises a container and a silicone gel disposed in the container.

6. The device of claim 1, wherein the cushion is formed around the second end of the support frame.

7. The device of claim 1, wherein the first end curves around a first axis and the second end curves around a second axis.

8. The device of claim 1, wherein the support frame and the cushion curves around a first axis.

9. The device of claim 1, wherein the support frame comprises a first arm having the first end and a second arm having the second end, wherein the first arm and the arm are connected at a bend of the support frame.

10. The device of claim 9, wherein the support frame is a metal band comprising the first arm and the second arm, wherein the bend positions the first arm and an angle with respect to the second arm of about 60° to about 120°.

11. The device of claim 1, wherein the spinal implant device comprises a plate having the first end and an arm having the second end and extending from a central portion of the plate, wherein the arm holds the cushion.

12. The device of claim 1, wherein the spinal implant device comprises a curved plane body having the first end and the second end, wherein more than one of the mounting hole is formed in the curved plane body.

13. A system for implanting a medical device to a spine, comprising:
a spinal implant device comprising:
a support frame, wherein the support frame comprises a first end, a second end, and a mounting hole; and
a cushion attached to the support frame; and
a fastener receivable through the mounting hole for fixably attaching the spinal implant device to a vertebra, wherein the spinal implant device is positionable to align the cushion to a disc space created after a portion of a herniated disc is removed during surgery.

14. The system of claim 13, wherein the cushion is positioned on the support frame to block expansion of the spinal disc when attached to the spine.

15. The system of claim 13, wherein the spinal implant device has a spring rate to maintain the position of the cushion in relation to the disc space during movement of the vertebra.

16. The system of claim 13 wherein the fastener comprises at least one of clips, screws, washers, nuts, bolts, pins, sockets, rods, studs, or adhesives.

17. The system of claim 13, wherein the cushion comprises a container and an incompressible fluid disposed in the container.

18. The system of claim 13, wherein the support frame comprises a first arm having the first end and a second arm having the second end, wherein the first arm and the arm are connected at a bend of the support frame.

19. A method for reducing the effects of a spinal disc herniation comprising:
removing a portion of a spinal disc to create a disc space;
positioning a spinal implant device against a vertebra adjacent to the spinal disc such that a cushion of the spinal implant device is aligned with the disc space, wherein the cushion is attached to a support frame of the spinal implant device; and
affixing the spinal implant device to the vertebra.

20. The method of claim 19, wherein affixing the spinal implant device comprises:
forming an attachment hole in the vertebra;
positioning the spinal implant device against an outer surface of the vertebra wherein the mounting hole is axially aligned with the attachment hole; and
affixing the spinal implant device to the vertebra with a fastener.
